(19) United States
(12) Patent Application Publication
(10) Pub. No.: US 2020/0205902 A1
(43) Pub. Date: Jul. 2, 2020

(54) METHOD AND APPARATUS FOR TROCAR-BASED STRUCTURED LIGHT APPLICATIONS

(71) Applicant: TransEnterix Surgical, Inc., Morrisville, NC (US)

(72) Inventor: Kevin Andrew Hufford, Cary, NC (US)

(21) Appl. No.: 16/010,388

(22) Filed: Jun. 15, 2018

Related U.S. Application Data
(60) Provisional application No. 62/520,554, filed on Jun. 15, 2017.
(60) Provisional application No. 62/520,552, filed on Jun. 15, 2017.

(51) Int. Cl. A61B 34/20; A61B 1/018; A61B 1/06; A61B 1/04; A61B 17/34

(52) U.S. Cl. CPC A61B 34/20; A61B 1/018; A61B 2034/302; A61B 1/04; A61B 17/3423; A61B 1/0661

(57) ABSTRACT

A method of using a surgical robotic system includes positioning a surgical instrument in a body cavity, the surgical instrument being one that is carried by a robotic arm. An image of an operative site within the body cavity is captured and used to identify a structure to be avoided. A user uses an input device to give input to the robotic system to cause movement of the surgical instrument at the site. The system determines determining whether the surgical instrument is approaching contact with the structure and, if it is, initiates an avoidance step.

This application claims the benefit of U.S. Provisional Application No. 62/520,552, filed Jun. 15, 2017, and U.S. Provisional Application No. 62/520,554, filed Jun. 15, 2017.

### Inventors: Kevin Andrew Hufford

### BACKGROUND

There are various types of surgical robotic systems on the market or under development. Some surgical robotic systems use a plurality of robotic arms. Each arm carries a surgical instrument, or the camera used to capture images from within the body for display on a monitor. Other surgical robotic systems use a single arm that carries a plurality of instruments and a camera that extend into the body via a single incision. Each of these types of robotic systems uses motors to position and/or orient the camera and instruments and to, where applicable, actuate the instruments. Typical configurations allow two or three instruments and the camera to be supported and manipulated by the system. Input to the system is generated based on input from a surgeon positioned at a master console, typically using input devices such as input handles and a foot pedal. Motion and actuation of the surgical instruments and the camera is controlled based on the user input. The image captured by the camera is shown on a display at the surgeon console. The console may be located patient-side, within the sterile field, or outside of the sterile field.

US Patent Publication US 2010/0094312 describes a surgical robotic system in which sensors are used to determine the forces that are being applied to the patient by the robotic surgical tools during use. This application describes the use of a 6 DOF force/torque sensor attached to a surgical robotic manipulator as a method for determining the haptic information needed to provide force feedback to the surgeon at the user interface. It describes a method of force estimation and a minimally invasive medical system, in particular a laparoscopic system, adapted to perform this method. As described, a robotic manipulator has an effector unit equipped with a six degrees-of-freedom (6-DOF or 6-axes) force/torque sensor. The effector unit is configured for holding a minimally invasive instrument mounted thereto. In normal use, a first end of the instrument is mounted to the effector unit of the robotic arm and the opposite, second end of the instrument (e.g. the instrument tip) is located beyond an external fulcrum (pivot point kinematic constraint) that limits the instrument in motion. In general, the fulcrum is located within an access port (e.g. the trocar) installed at an incision in the body of a patient, e.g. in the abdominal wall. A position of the instrument relative to the fulcrum is determined. This step includes continuously updating the insertion depth of the instrument or the distance between the (reference frame of the) sensor and the fulcrum. Using the 6 DOF force/torque sensor, a force and a torque exerted onto the effector unit by the first end of the instrument are measured. Using the principle of superposition, an estimate of a force exerted onto the second end of the instrument based on the determined position is calculated. The forces are communicated to the surgeon in the form of tactile haptic feedback at the hand controllers of the surgeon console.

Often in surgery there are tissues within the body cavity that the surgeon would like to avoid touching with the surgical instruments. Examples of such structures include the ureter, nerves, blood vessels, ducts etc. The need to avoid certain structures is present both in open surgery, as well as in the domain of laparoscopic surgery, including minimally-invasive gynecologic, colorectal, oncologic, pediatric, urologic, or thoracic procedures, as well as other minimally-invasive procedures. The present application describes features and methods for improving on robotic systems by allowing control of the robotic system based on information about identified tissues or structures within the surgical field. They may also be more generally used to assist with tasks or guide tasks.

Embodiments described below include the use of data generated using structured light techniques performed by illuminating the body cavity using structured light delivered from a trocar through which the surgical instrument is inserted into the body.

### BRIEF DESCRIPTION OF THE DRAWINGS

FIG. 1 is a block diagram schematically illustrating the function of the disclosed system and method.

FIG. 2 schematically illustrates a first embodiment making use of an endoscope image as the information source.

FIG. 3 schematically illustrates a second embodiment making use of an endoscope image as the information source, in combination with the use of motion prediction based on the endoscope image.

FIG. 4 schematically illustrates a third embodiment making use of endoscope image and arm information as the information sources.

FIG. 5 schematically illustrates a fourth embodiment making use of an endoscope image, other imaging sources, plus arm and surgeon input.

FIGS. 6-9 illustrate use of computer vision to identify an instrument and its location, as well as a ureteral stent disposed in a ureter, and the incorporating of the poses of the instrument and stent into a model.

FIG. 10 gives one example of the timing and frequency of the availability of different types of information to the system.

FIG. 12 is a side elevation view of a second embodiment of a trocar for trocar-based structured light applications.

FIG. 13 is a side elevation view of a third embodiment of a trocar for trocar-based structured light applications.

### DETAILED DESCRIPTION

The present application describes a system and method that make use of information provided to the system about the operative site to allow the robotic surgical system to operate in a manner that avoids unintended contact between surgical instruments and certain tissues or structures within the body. These features and methods allow the system to track the identified structures or tissues and predict whether the instrument is approaching unintentional contact with the tissue or structure to be avoided. Such features and techniques can help protect delicate tissues by automatically controlling the robotic system in a manner that stops or prevents the unintentional contact and/or that gives feedback to the surgeon about the imminence of such contact as predicted by the system so that the surgeon can avoid the predicted contact. They may also be more generally used to assist with tasks or guide tasks. In some cases, the system may be used to track other structures placed in the body, such as ureteral stents (which can help to mark the ureter so it may be avoided during the procedure), or colpotomy cups.

Some embodiments described below also include the use of data generated using structured light techniques performed by illuminating the body cavity using structured light delivered from a trocar through which the surgical instrument is inserted into the body.

Structures/tissues that are identified and/or tracked may be ones that fluoresce, whether by autofluorescence, using a fluorescent agent such as indocyanine green (ICG) or a dye such as methylene blue.

The surgical system may be of a type described in the Background, or any other type of robotic system used to maneuver surgical instruments at an operative site within the body.

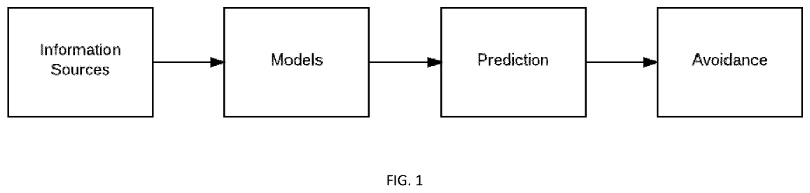

At a high level, embodiments described in this application provide method of controlling a robotic surgical system based on identified structures, such as those identified within an endoscopic camera image. Some implementations use additional data sources to provide anticipatory information. The invention acquires data from a source or number of sources, processes that information, and provides output to the surgeon based on that information. As indicated in FIG. 1, the system amalgamates information and processes it to provide actionable data to improve control of the robotic system.

Some embodiments identify structures and provide control input to a robotic surgical system with a limited amount of information. In other embodiments, a richer set of information provides additional benefits, which may include a more responsive system, a system that is easier to use, and others.

The invention may be implemented in a number of ways by incorporating various layers of information. These may include, but are not limited to the following:

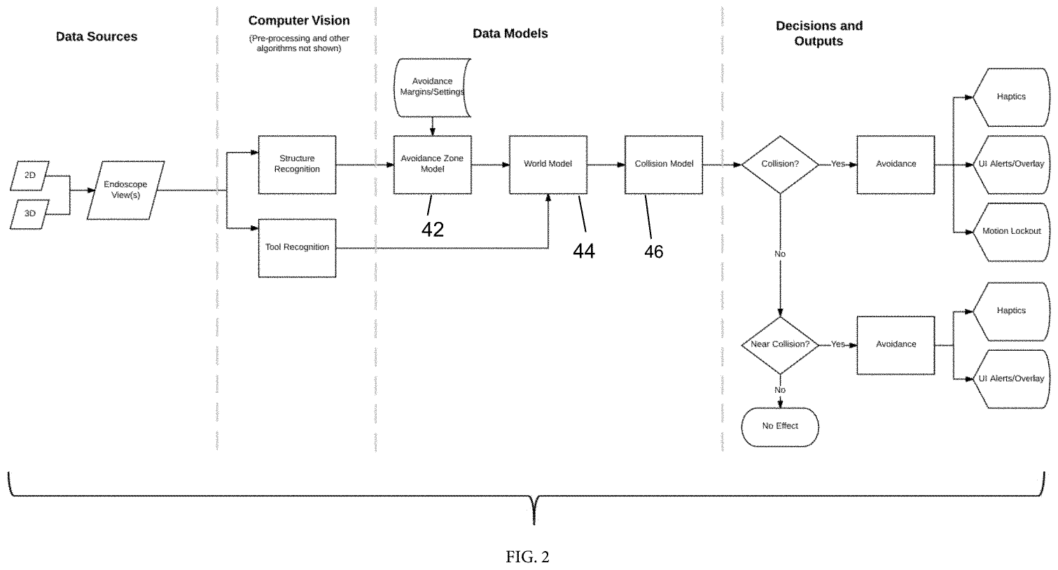

Endoscope Image only (FIG. 2)

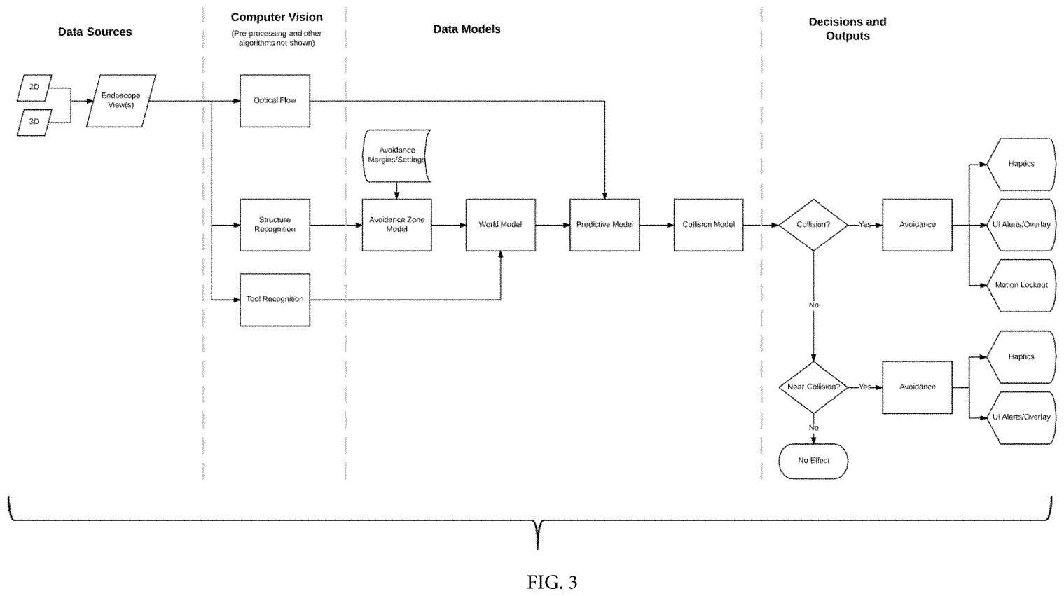

Endoscope Image+Motion Prediction on the Endoscope Image (FIG. 3)

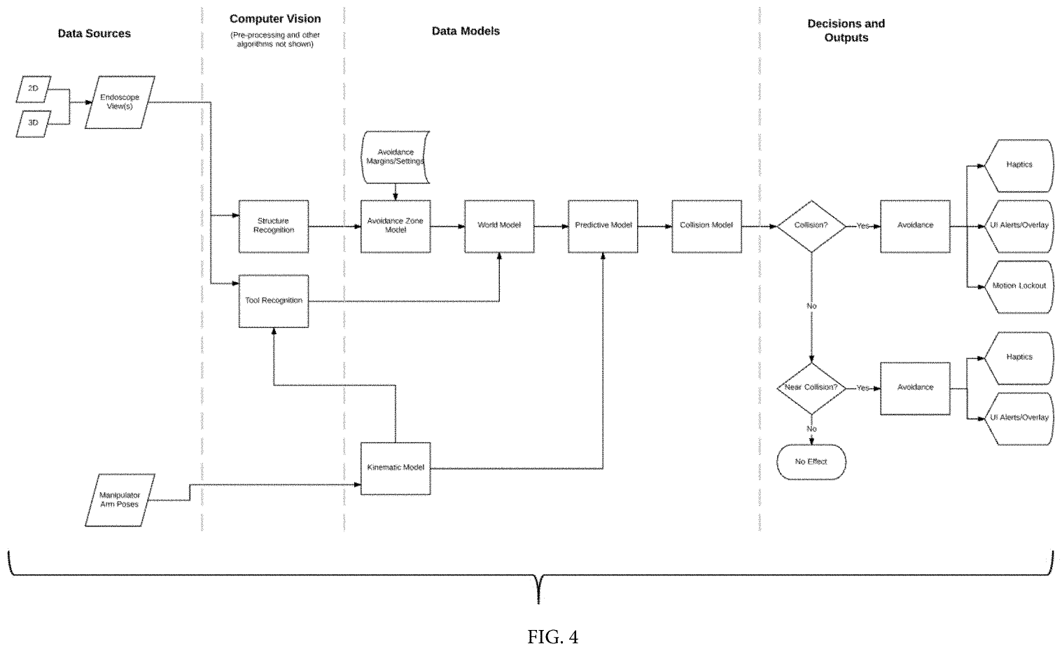

Endoscope Image+Arm Information Only (FIG. 4)

Endoscope Image+Arm+Surgeon Input

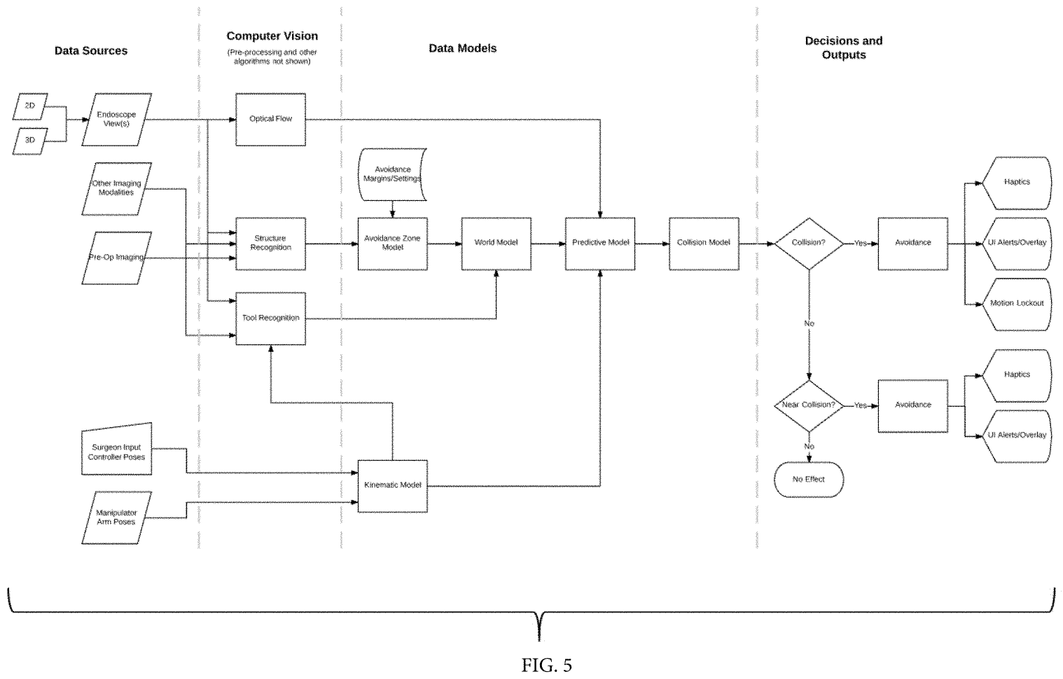

Endoscope Image+Other Imaging Sources+Arm+Surgeon Input (FIG. 5)

Referring to FIG. 2, in a first embodiment, data sources are used to input information to the system about the operative site. As one example, a 2D and/or 3D camera captures views of the operative site. Computer vision techniques are applied to the image data to recognize tissues/structures within the body cavity that are of interest to the surgical staff, and particularly those that the surgeon wishes to avoid contacting with the surgical instruments. User input may be given to instruct the surgeon as to what tissues/structures within the operative site are to be avoided. For example, the user might use an input device to navigate an icon or pointer to a structure or tissue region visible on the display, or to highlight tissue within a certain bounded area or lying at a particular tissue plane (e.g. a tissue plane identified using structured light techniques), and to then input to the system that the marked tissue/structure should be avoided. In other implementations, the computer vision algorithm automatically recognizes the instruments and/or the structures. Computer vision techniques are similarly used to recognize the surgical instruments/tools within the operative site.

The system makes use of several data models as shown in FIG. 2. A first model is an Avoidance Zone Model, which is based on data representing the identified structure (in 2 or 3 dimensions) and system settings including those corresponding to the avoidance margin (i.e. by how far should the instrument avoid contacting the tissue). A second model is a World Model, a spatial layout of the environment within the body cavity created based on the location of the tissues/structures to be avoided (from the Avoidance Zone model), and the tool position and pose. A Collision Model takes into account the avoidance zone, the tool position/pose, as well as other information. Based on the Collision Model, the system determines whether a collision is occurring and/or whether a collision is near. If a collision is occurring, avoidance steps may be taken such as providing haptic feedback (rigidity, a gentle push away from a boundary, vibrational input, etc.) to the user and the user input controls, providing other alerts to the user such as visual overlays on the display showing the camera image, auditory alerts, etc, stopping further motion of the surgical instrument within the body cavity, and/or the prevention of motion of the system beyond a certain point or in a direction or series of directions/orientations.

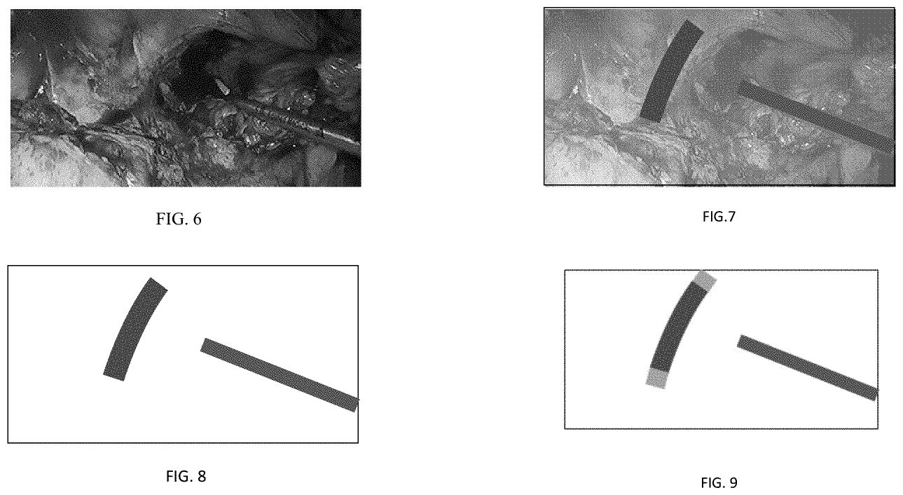

Input of information into the data models is illustrated in FIGS. 6-9. FIG. 6 shows an image from a laparoscopic camera showing an instrument along with a ureteral stent disposed within a ureter under layers of tissue. FIG. 7 shows the image of FIG. 6, with visual indicia indicating that a computer vision algorithm has identified the instrument and its location, as well as the lighted ureteral stent. As indicated in FIG. 8, the poses of the instrument and stent are input into a model. In some cases, the computer vision system can recognize structures or further extents of structures (e.g. a portion of an instrument more deeply positioned within tissue than portions visible on the camera display) that are not visible to the surgeon. The affects of various wavelengths of light penetrating through tissue may be used to extract depth information about such structures. In the case of a lighted ureteral stent, for instance, the wavelength(s) are known. It may be possible to transmit various wavelengths, a pattern, or strobe pattern, and use that to determine the stent's presence and, potentially, its depth. This allows identification of the depth/positional information of a structure based on transmitted spectral information.

As discussed above, to aid the computer vision algorithm in image segmentation and improve robustness, user input may be used to select or guide the algorithm. The user may be prompted to select the tip of the instrument, or “click on the lighted ureter”. This may be with a mouse, touchscreen, the hand controllers, or other input device. In some implementations, eye tracking is used to provide user input.

While the embodiment of FIG. 2 makes use solely of the camera image to create the model of the environment, additional imaging sources may help to enhance the model of the environment as is reflected in FIG. 5. Additional sources may be incorporated into any of the illustrated embodiments. Such additional sources may include pre-operative images, such as MRI or CT images. In some cases, a peri-operative CT or ultrasound may be taken, and may be co-registered to or tracked by an optical tracking system, or by the robotic surgical system. These image sources may be static, or may be dynamic. Dynamic sources of imaging may include, but are not limited to: ultrasound, OCT, and structured light. Any combination of sources may be used to create a model of the anatomy, which then may be constructed as a deformable model that updates based on the live/real-time/near real-time imaging sources. This may update boundaries/tissue planes that should not be violated, for instance.

In a second embodiment schematically shown in FIG. 3 incorporated motion prediction based on the endoscope image. Optical flow is a technique that is used for assessing motion in video images. These algorithms recognize and track the motion of points within the image, providing provides direction vectors that describe the motion of a pixel (or group of pixels or object) between frames. In the FIG. 3 embodiment, optical flow algorithms are used to provide some predictive information from the endoscope image that aids in the determination of whether a collision is expected to occur.

In a third embodiment shown in FIG. 4, a predictive algorithm uses the actual position of the robotic arm to provide anticipatory information of where the tool tip may be in the endoscopic image. In a fourth embodiment shown in FIG. 5, the predictive algorithm uses the input from the surgeon console as well as the actual position of the robotic arm to provide anticipatory information of where the tool tip may be in the endoscopic image. See, FIG. 5. As with the embodiment of FIG. 3, the predictive algorithms of these embodiments aid in the determination of whether a collision is near.

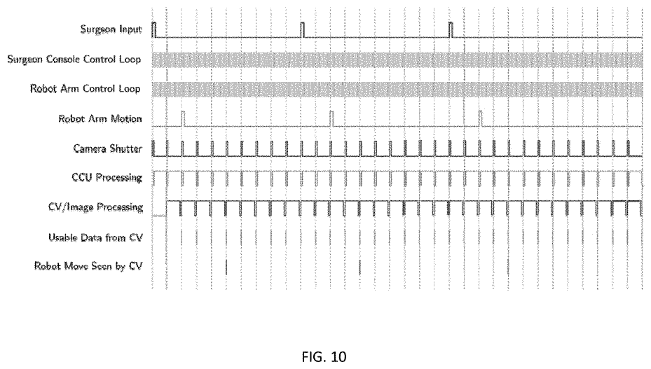

The information used by the system may be provided to the system or updated at different time intervals. For instance, a camera image may be available at approximately 30 Hz or approximately 60 Hz. Less frequently, an endoscopic image may be available at approximately 50 Hz. In contrast, the control loop and resultant information for a surgical robotic system may be at 250 Hz, 500 Hz, 1 kHz, or 2 kHz. See FIG. 10, which shows an example of the timing of the availability of these types of information.

This presents an opportunity for using higher-fidelity information, but it is necessary to rectify the timing of information coming from different sources.

In FIG. 10, an endoscopic image at 30 Hz is shown. A robotic system latency of ˜60 ms is shown. After CCU processing and CV/Image processing, the motion may be only detected after >60 ms have passed, and >120 ms after the surgeon initiated the motion. Based on this information, avoidance methods may be used and/or feedback given to the surgeon.

As discussed above, additional imaging sources may help to enhance the model of the environment. These imaging sources may be co-registered to or tracked by an optical tracking system, or by the robotic surgical system. These image sources may be static, or may be dynamic. Dynamic sources of imaging may include, but are not limited to: ultrasound, OCT, and structured light. Any combination of sources may be used to create a model of the anatomy, which then may be constructed as a deformable model that updates based on the live/real-time/near real-time imaging sources. This may update boundaries/tissue planes that should not be violated, for instance.

A source of structured light may be used to generate additional information in any of the embodiments described above. In some implementations, a source of structured light may be added to the trocar through which the surgical instrument is inserted into the body. This may be an optical element/series of optical elements, or a light source and optical element/series of optical elements. In some implementations, an external light source may be connected (by attachment, by simple proximity, by fiber optic connector, etc.) to the component that provides structured light.

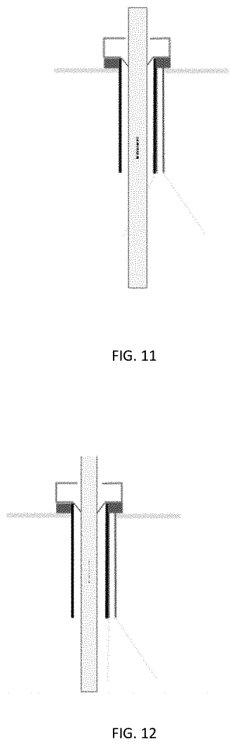

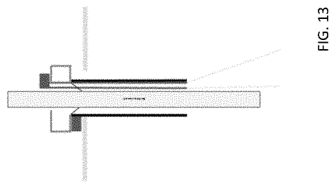

In some implementations, the light source/optical element is outside the nominal circumference of the trocar as shown in FIG. 11. In others, the source of structured light may not project an image that is axisymmetric with the trocar or the tool, as shown in FIG. 12. In some implementations, such as the one shown in FIG. 13, the light source/optical element is inside the nominal diameter of the trocar. Multiple sources of structured light may be used to minimize occlusions from a surgical tool or other obstacles.

In some implementations, the optical element and/or light source for providing the structured light may be on a sliding/movable element that moves along with the insertion of the instrument. This may allow the structured light source to be closer to the tissue or to maintain a constant/optimal distance.

In some implementations, a source of structured light may be integrated into the trocar.

In some implementations, part of the optical path may be the trocar lumen itself. In some implementations, part of the optical path may be features molded into the surface or structure of the trocar lumen. Alternative implementations may be features attached to or machined/etched/post-processed into the surface or structure of the trocar lumen.

In some implementations, the trocar lumen structure may be overmolded onto optical elements.

The following is a sequence of steps in an exemplary method for providing the illumination:

1. The structured light source ring is attached to the trocar

2. The skin incision/insertion of the Veress needle is performed per standard procedure/surgeon preference.

3. The trocar with structured light source is inserted.

The text accompanying FIG. 10 described the timing of information availability for various sources. In some implementations, the structured light is synchronized with the endoscopic camera image. This may alternate frames with a normally-illuminated camera image, or have alternate timings. The structured light may alternately be an infrared source, in which case alternate filters may be used on elements in the camera array as, and alternating between frames with normal-illumination and frames used for structured light may not be necessary.

As also referenced above, optical flow/motion algorithms may be used to provide predictive motion for tissue positions and/or tool positions. Based on this information, avoidance methods may be used and/or feedback given to the surgeon.

In an alternate embodiment, a source of structured light that is attached to the abdominal wall may be used. In some implementations, this may be magnetically held; potentially with an external magnetic or ferrous device outside the body.

The invention(s) are not limited to the order of operations shown and may not require all elements shown; different combinations are still within scope of the invention. use of transmitted spectral information to determine the depth of an identified structure.

All prior patents and applications referred to herein, including for purposes of priority, are incorporated herein by reference.

We claim:

1. A method of using a surgical robotic system, comprising the steps of:
positioning a surgical instrument in a body cavity, the surgical instrument carried by a robotic arm;
capturing an image of an operative site within the body cavity;
using the image, identifying a structure to be avoided;
using an input device to give input to the robotic system to cause movement of the surgical instrument at the site;
automatically determining whether the surgical instrument is approaching contact with the structure; and
initiating an avoidance step if the system determines the surgical instrument is approaching contact with the structure.

2. The method according to claim 1, wherein identifying the structure includes using structured light techniques.

3. The method according to claim 2, wherein the structured light techniques are carried out using structured light illumination of the body cavity using light sources on a trocar.

4. A method of identifying structures within a body cavity using structured light techniques, the structured light techniques are carried out using structured light illumination using light sources on a trocar,

5. A trocar adapted to illuminate body tissue in a pattern for use in structured light techniques.
